(19) United States
(12) Patent Application Publication
(10) Pub. No.: US 2005/0246106 A1
(43) Pub. Date: Nov. 3, 2005

(54) Methods and systems for identifying genes, splice variants, and transcripts using an evidence mapping approach

(75) Inventors: Chunlin Xiao, Rockville, MD (US); Valentina Di Francesco, Rockville, MD (US); Brian P. Walenz, Rockville, MD (US); Peter W. Li, Brookeville, MD (US); Michael J. Campbell, San Francisco, CA (US); Liliana Florea, Washington, DC (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/945,597

(22) Filed: Sep. 20, 2004

Related U.S. Application Data
(60) Provisional application No. 60/504,309, filed on Sep. 18, 2003.

(51) Int. Cl. C12Q 1/68; G06F 19/00; G01N 33/48; G01N 33/50

(52) U.S. Cl. 702/20; 435/6

(57) ABSTRACT

The present teachings contain a method and system for gene and splice variant identification and display. The teachings employ evidence from various genomic and proteomic databases. The method can be run on a computer system and performs its function by mapping evidence to a genome under study and then collapsing overlapping evidence to form clusters. Various rules are taught that can be used to identify unique transcripts and gene boundaries. Clusters can be displayed visually or in report format. Regardless of format, the evidence leading the cluster definitions can be seen.

### CROSS-REFERENCE TO RELATED APPLICATIONS

This application claims priority to U.S. Provisional Patent Application No. 60/504309, filed on Sep. 18, 2003, which is incorporated herein in its entirety by reference.

### FIELD

The present teachings relate to the field of gene, alternative slice variant and transcript identification and display.

### INTRODUCTION

Gene prediction programs are generally based on ab initio homology principles. In general, the advantage of these prediction programs is their ability to provide in silico genes on either incomplete or complete genomes with limited experimental evidence—so as long as the predicted genes follow predefined models. Unfortunately these programs often produce gene locations and structures of low confidence and/or high rates of over/under prediction.

Accurate identification of genes typically requires manual curation. This involves merging diverse sources of information such as the output of different gene prediction programs, homology searches of proteins and sequence databases. The process is often time consuming and error prone. Differences in the manner in which individual curators attack the problem can also produce inconsistent results where one curator may identify gene boundaries differently than another. The present teachings discuss computational methods that can relieve a human operator from this burdensome task. They can identify and annotate genes, their variants, and identify transcripts in an accurate and consistent manner. This information can be useful for basic, biomedical, and pharmaceutical research.

### DESCRIPTION OF THE DRAWINGS

The skilled artisan will understand that the drawings, described below, are for illustration purposes only. The drawings are not intended to limit the scope of the present teachings in any way.

Figure one gives the number of sequences in various sequence databases.

Figure two illustrates an embodiment of the present teachings that can be used for gene, splice variant and transcript identification.

Figure three illustrates an embodiment of the present teachings that graphically displays cluster information.

Figure four illustrates a comparison of splice junction differences between curated Celera transcripts and (a) non-curated Celera transcripts, (b) Mammalian Genome Collection transcripts, and (c) GenBank mRNAs.

Figure five represents a (a) histogram of unique exon lengths for Celera transcripts (b) histogram of unique exon lengths for Celera transcripts, and (c) histogram of the shortest exons per Celera transcript.

Figure six illustrates the comparison of intron structure between sequences.

Figure seven illustrates an embodiment of the present teachings that (a) graphically displays cluster information, and (b) allows a user to input sequence identifiers to be located in clusters.

Figure eight shows the mapping rate of various evidence database sequences to the human genome.

Figure nine shows results for collapsing overlapping ESTs into clusters.

Figure ten illustrates clone-end alignment information for individual ESTs observed during EST clustering.

Figure eleven shows the number of clusters identified by an embodiment of the present teachings via different combinations of evidence.

Figure twelve compares the number of transcribed genes determined by an embodiment of the present teachings to the number of genes identified by the Ensembl and RefSeq organizations

Figure thirteen is a Venn diagram illustrating the overlap in gene identification between the transcribed genes determined by an embodiment of the present teachings to those genes identified by the Ensembl and RefSeq organizations.

Figure fourteen illustrates the types of clusters encountered when clustering curated Celera transcripts.

Figure fifteen shows the number of clusters identified by an embodiment of the present teachings via different combinations of evidence.

Figure sixteen illustrates an embodiment of a computer system upon which various embodiments of the present teachings can be implemented.

### DESCRIPTION

The section headings used herein are for organizational purposes only and are not to be construed as limiting the subject matter described in any way.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Figure one shows the number of genes estimated by various research organizations. This figure shows that the number of genes varies widely in the literature and emphasizes that current gene prediction tools are imperfect in identifying correct genes/transcripts and splicing variant. Experimental sequences expressed from human genes can be extremely useful in identifying and annotating human genes. The present teachings discuss a genome-based-evidence-clustering approach that focuses on genetic information such as, genes, splice variants, and transcripts on a genome using evidence. Such evidence can include information from the Mammalian Gene Collection (MGC), GenBank mRNAs, ESTs, and RefSeq NMs.

Figure two illustrates an embodiment of the present teachings identifies genetic information using evidence from various sources to build clusters. In 202, observed information is collected from various data sources. A variety of data sources can be used as input. For example, two high quality sources that have been manually reviewed by biologists, and are therefore thought to be complete are the Celera Transcripts (CTs) and the RefSeqs (NMs). Another class of sequences is the Mammalian Genome Collection sequences (MGCs). These sequences arise from the end-to-end sequencing of cDNAs from many human tissues. An an entire protein coding region must be found in order that a full-sequenced cDNA be promoted to an MGC. Another class of sequences are GenBank mRNAs which have no quality standards associated with them other than encouraging people to deposit only full-length sequences. A somewhat less reliable data source is the set of ESTs (expressed sequence tagged). These are transcript fragments. EST sequences are generated from single-pass reads and can have a potentially high rate of sequencing errors, contamination from vectors, retroviruses or genomic amplification. Additionally protein information can be used. This information can be mapped to the genome using programs such as GeneWise. One skilled in the art will appreciate that many other data sources can be used and that the teachings herein are not limited to the sources mentioned above.

At 204, the data sources are mapped to the genome. This mapping can be performed via a variety of techniques. Some embodiments use programs such as SIM4 (Florea et. al., A computer program for aligning a cDNA sequence with a genomic DNA sequence, Genome Research 8(9):967-74) which is incorporated by reference herein for all purposes or BLAT (Kent, Genome Research 12(6):656-664). At 206, the evidence can be ranked in terms of its quality. For example, the mapped sequences can be ranked according to a variety of metrics including the percentage of the sequence aligned, the amount of coverage, the numbers of exons spanned and the number of cDNA gaps. Some embodiments retain only evidence that surpass a user-defined quality measure. For example, only evidence with 98% identity and 50% coverage may be retained. Ranking and filtering data in this way can provide a high-quality result.

At 208 the evidence is collapsed into clusters. Some embodiments create clusters that define regions of transcriptional activity and can define gene boundaries. Some embodiments use intron spacing information to identify splice variants.

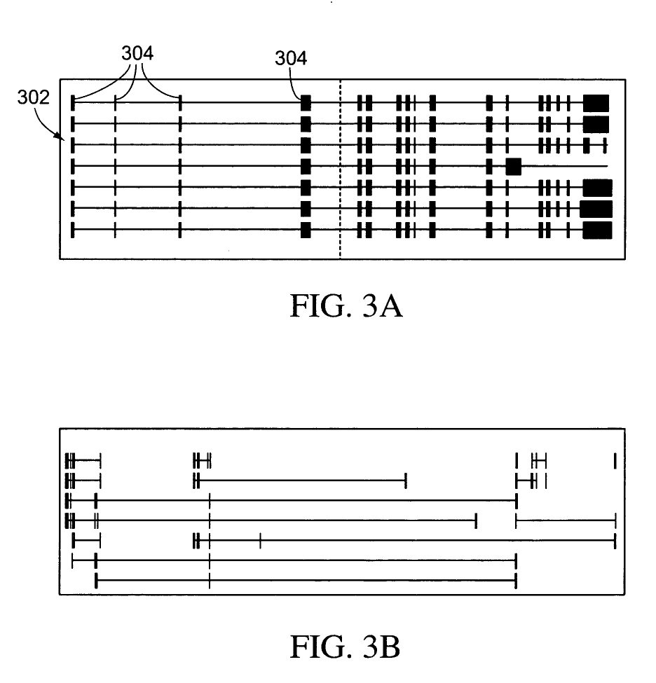

At 210, the clusters are output. This output can take a variety of forms such as a report or a visual representation. FIG. 3 illustrates an embodiment of a visual representation of the clustering results. Here, the horizontal lines (302) represent the clusters with thicker regions (304) indicating the exons. In FIG. 3a the various clusters line up precisely and suggest that only one gene is present in that region. In FIG. 3b several cluster lie within a region and since they do not compress into a single structure, the figure suggests the region may have multiple genes. Various embodiments allow the user to select a cluster and view the underlying evidence.

Identification of Gene Boundaries

Various embodiments identify gene boundaries via clustering at 208. This can be accomplished by determining the relationships of all sequences selected earlier. Some embodiments establish these relationships by performing pair-wise and all-against-all sequence comparison and then examining sequence characteristics such as a sequence's genomic location, matching identities and coverage on the reference genome, numbers of exons and introns, overlapping types (exon or intron overlap), overlapping lengths, percentages of overlapping length, the numbers of overlapped exons, or the numbers of exactly-matched splicing sites given any two selected sequences. Given this information, various embodiments define a gene model. This can be accomplished by establishing a criterion which, when met, establishes that two sequences belong to the same gene (called gene-links.) In some cases the criteria is that at least one overlapping exon on the same strand of the same genome location is shared between two sequences. One skilled in the art will appreciate that other criteria can be used and/or parameters, such as the amount of overlap, adjusted depending on the level of stringency desired. Some embodiments further cluster by identifying all sequences that are linked to another sequence by a gene-link. These sequences can then be assigned to a single cluster. In some instances, if no gene-link exists for a given sequence, the sequence itself can form a singleton cluster.

Various embodiments split clusters if two or more well-defined RefSeq loci are linked through non-RefSeq evidence sequences. In this case, overlapped sequences can be assigned to appropriate clusters according to their mapping qualities.

The boundary of a given cluster can be defined as the maximum span of all evidence sequences that form the cluster. In some embodiments, if a cluster contains a known gene such as a RefSeq locus, then the boundary of the cluster can be referred to as a gene boundary.

Identification of Splice Variants

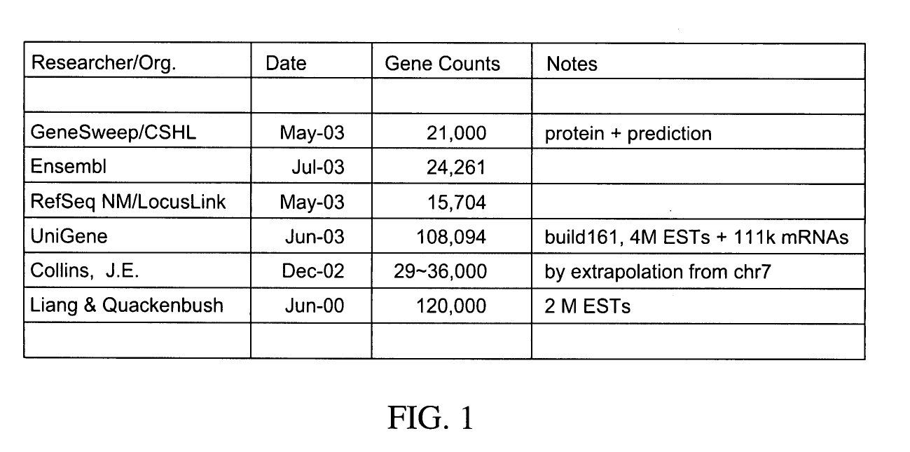

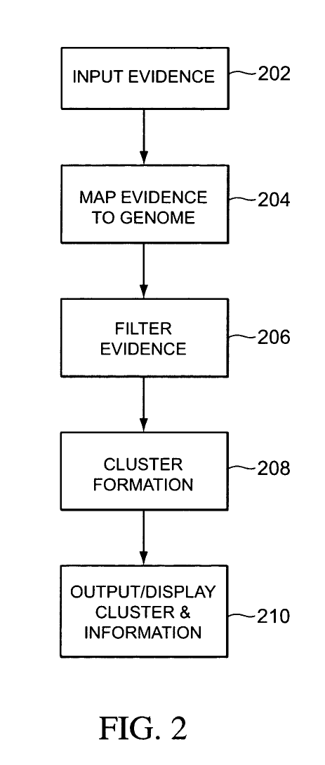

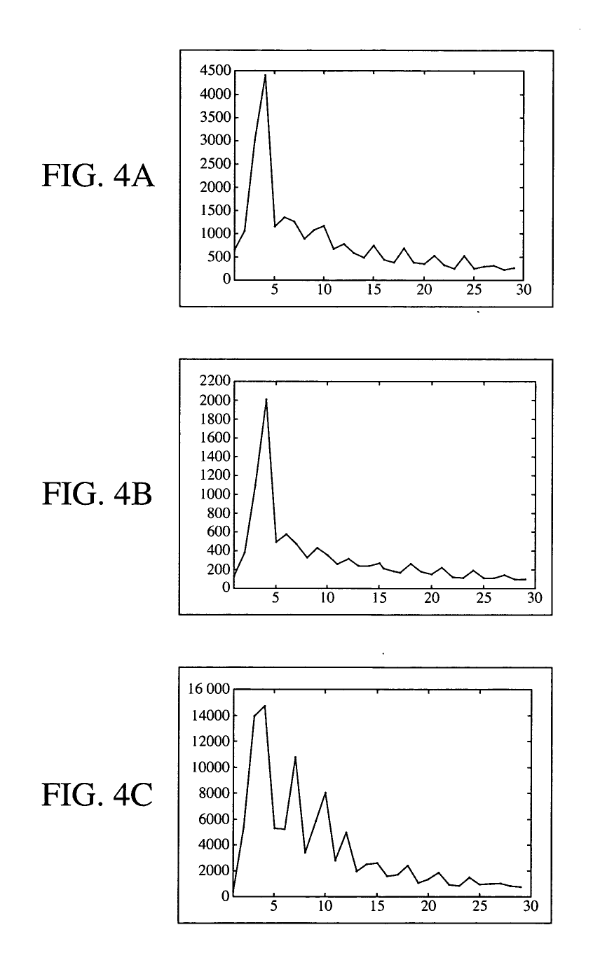

Various embodiments identify splice variants at 208 in FIG. 2. Because splicing always involves the removal of introns, except in the case of a single exon gene, clusters can be formed by determining if sequences possess similar intron structure. Similar intron structure can be defined as the possession of the same number of introns and the same and stop positions within some reasonable user-defined threshold. Various embodiments, also consider whether the shorter two sequences being compared can be considered full length or, lacking enough confidence to be considered full length, partial length. Because splice junctions can vary, a user-defined threshold can be used to determine if splice junctions between sequences are equivalent. An effective criterion for splice junction similarity can be determined by aligning high-quality independently generated sequences and examining how often and by how much splice junctions vary. For example, curated Celera transcripts, non-curated Celera transcripts, Mammalian Genome Collection (MCG) transcripts and GenBank mRNAs were examined and it was determined that between 98.2% and 98.5% of splice junction positions occur at exactly the same position. Histograms of those splice sites that do vary are shown in FIG. 4 where curated Celera transcripts are compared to non-curated Celera transcripts in FIG. 4a, Mammalian Genome Collection (MCG) transcripts in FIG. 4b and GenBank mRNAs in FIG. 4c. In these figures, nearly all differences occur within 15 bp of each sequence's end, with a significant spike at approximately 4 base pairs.

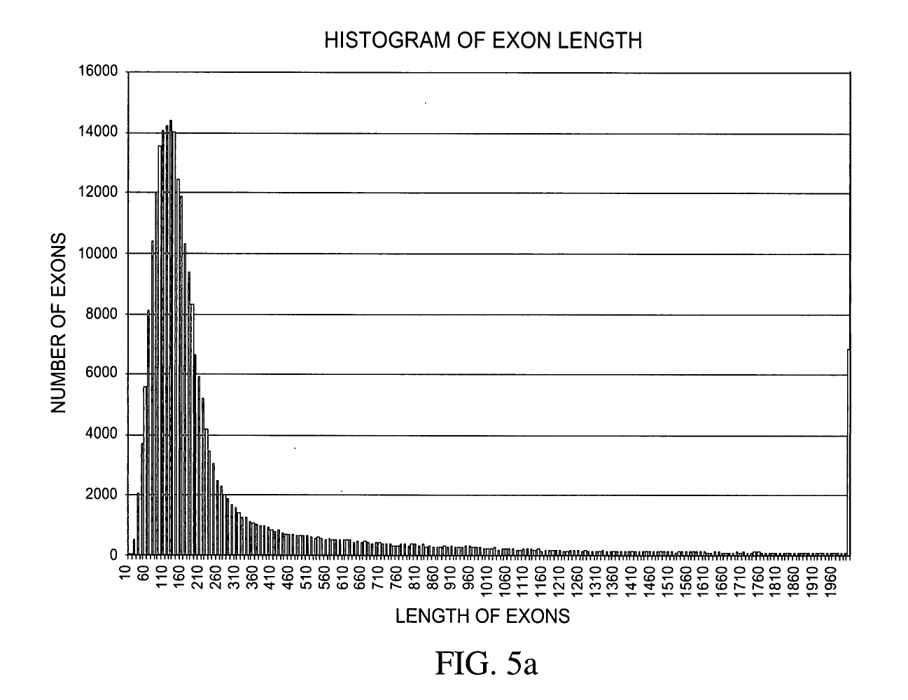

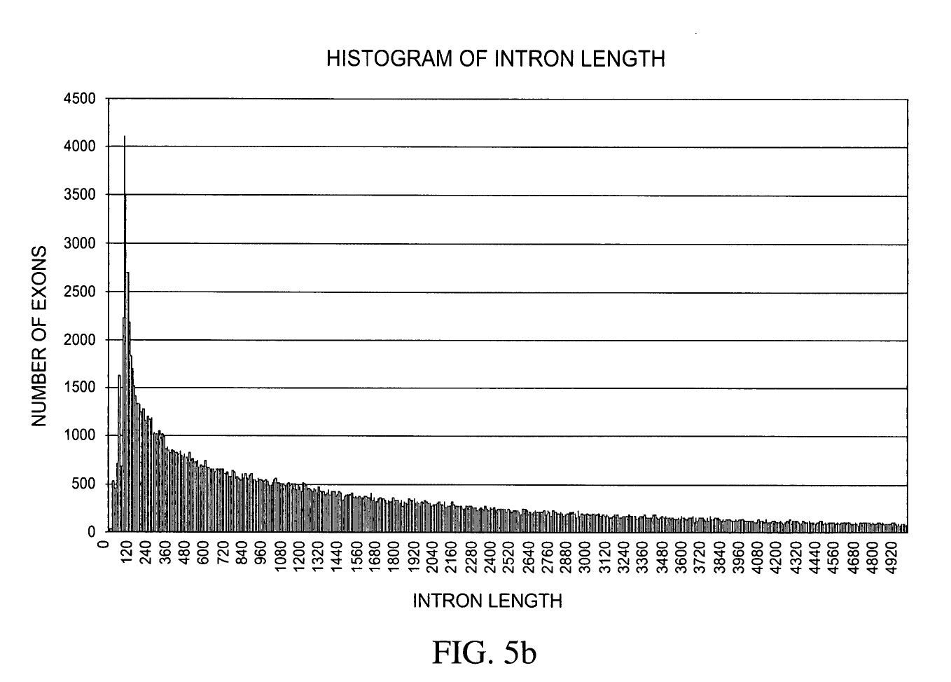

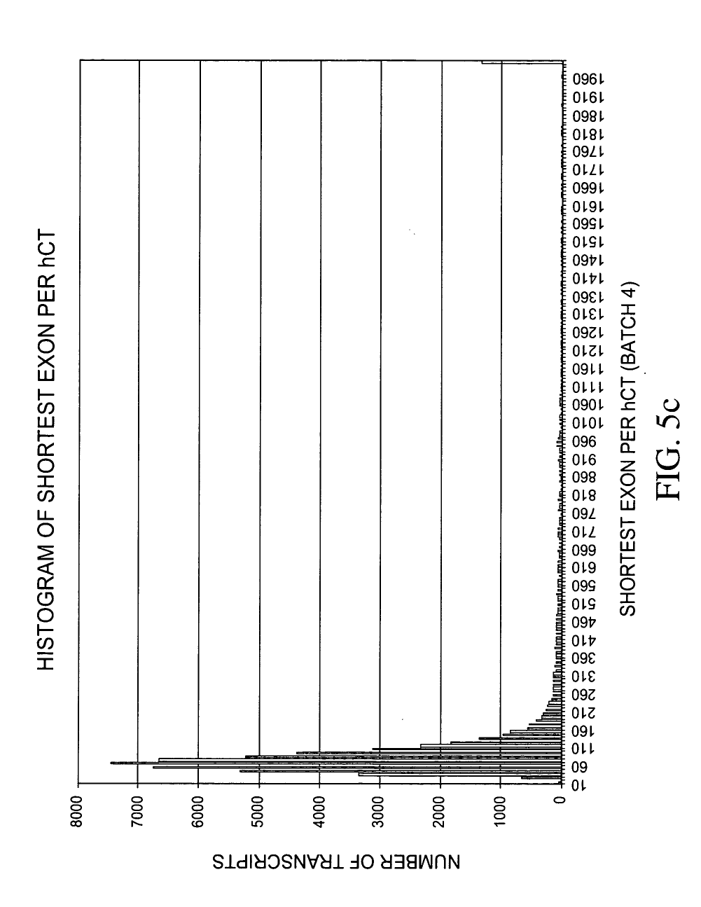

The impact of the user-defined threshold on short sequence exclusion can be evaluated by determining how frequently introns and exons are below the cutoff. This can be accomplished via the histograms contained in FIGS. 5a and 5b which histogram human Celera transcripts (hCTs) unique exon and intron lengths respectively. These figures show that less than 1% of all curated human Celera Transcripts have introns or exons less or equal to 15 base pairs. FIG. 5c shows that the number of exons per hCT below the cut-off almost triples if the cut-off is increased to 20 base pairs. Thus, some embodiments use a cut-off of 15 base pairs to determine if two segments have the same end point however, one skilled in the art will appreciate that other values can be used.

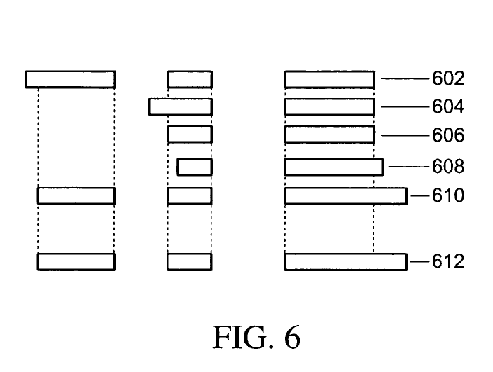

FIG. 6 illustrates how an embodiment of the present teachings clusters sequences. One consideration can be if the sequence is considered full or partial length. For example, both 602 and 612 will cluster together regardless of whether or not each sequence is considered full-length or partial-length because they possess identical intron structure. As another example, if sequence 602 is thought to be full length, it can be used as a reference sequence. The four transcripts labeled 604, 606, 608, and 610 have a different number of introns than 602, but agree with the 602 wherever they have introns. If these sequences are considered partial sequences, they will cluster with the reference. If they are of high confidence (full length), they will cluster into separate transcripts. Because the introns of partial-length sequences may match with the intron structures of more than one transcript cluster, such sequences can be assigned to all clusters that they are consistent with. This can be permitted because if a sequence is lacking in some of its information, it is difficult to say which form it belongs to. Likewise, any single-exon partial-length sequences can be allowed to cluster with multiple transcripts.

In some embodiments the ends of a sequence are not used in the clustering process. Because of this, the end of a partial-length sequence does not need to extend all the way to the next intron-exon boundary of the longer sequence it is matched to. For example, in FIG. 6, 606 does not extend to the left-most intron however, 604 does extends into the left-most intron. Regardless, they will cluster together. Some embodiments use the rule that partial-length sequences must have a novel set of introns not found elsewhere to create a new transcript cluster. In such a situation, if sequences 604 and 606 are partial length, they will cluster with the reference sequence. Various embodiments will still cluster single-exon partial-length sequences as long as they overlap an exon of another cluster by 1 bp or more. This can permit the user to screen out poor quality information that is contained in any of the reference databases.

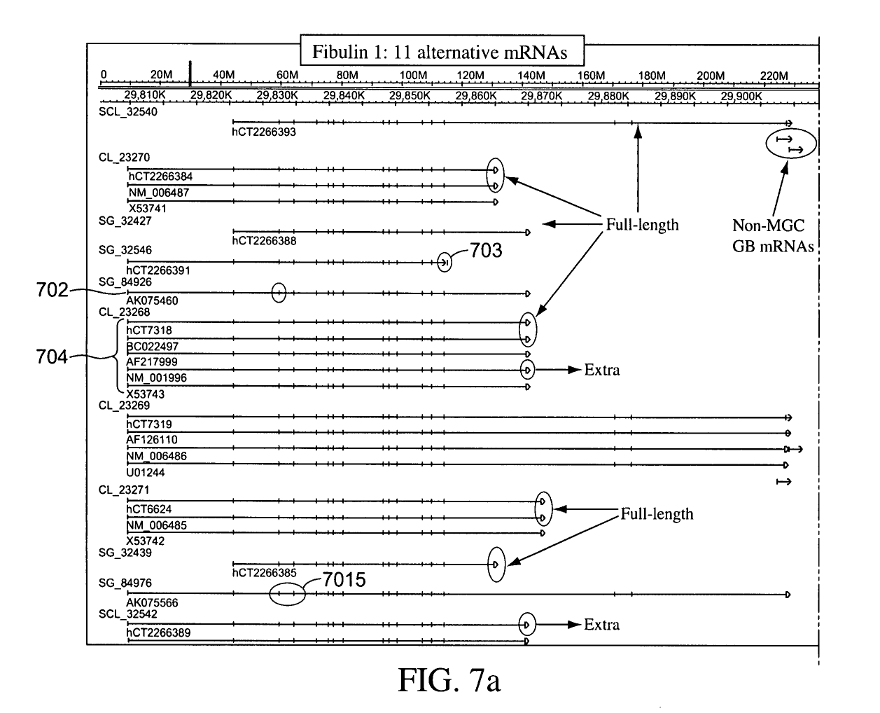

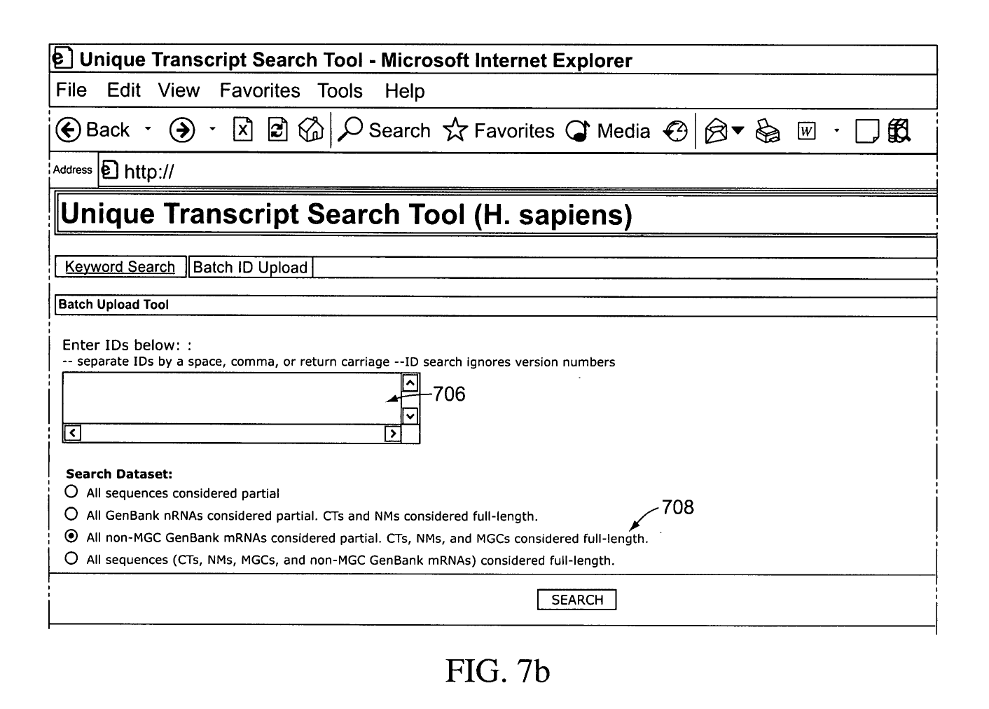

Some embodiments provide a method of viewing the clusters. FIG. 7a illustrates an embodiment of such a viewer implemented on a computer display. Element 702 shows the result of a singleton cluster. In this instance no evidence was located that mapped to the reference sequence and because a high-quality curated sequence set was used as the reference, and the assumption that the sequences contained in this set are full length, the transcript forms a singleton cluster. However element 704 illustrates that several pieces of evidence were used in defining the cluster. As in FIG. 3, the horizontal lines, such as 702, represent the transcripts, the thicker portions of the line indicates the exons and the thinner portion represent the introns. One skilled in the art will appreciate that they are a variety of ways that the data can be represented and the evidence leading to a cluster can be shown. The viewer can be zoomable so that portions of the figure can be inspected in detail. For example 705 shows a novel exon that when zoomed out may not be distinguishable but upon zooming, will be revealed to be in a different locus that other transcripts. Elements 703 highlights other novel exons. FIG. 7b illustrates an embodiment of the present teachings that permits search functionality. The user can input one or more evidence identifiers for a piece of evidence at 706, and optionally choose from various clustering datasets that can be based on different sets of rules (708.) Upon searching, a viewer such as the viewer in 7a is displayed that shows the clusters to which the sequence named by the one or more evidence identifiers belong. Various other embodiments display on the viewer assay products for sale that can assist the researcher in identifying a given transcript cluster in a sample. These assays can be designed and provided to the user via methods that are taught in PCT Application Publication Numbers WO03/64694 and WO03/65146 which are incorporated herein by reference in their entirety.

### EXAMPLE 1

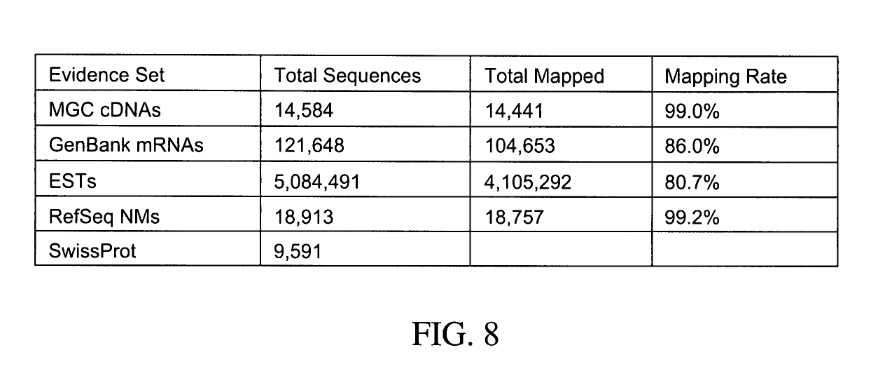

FIG. 8 shows results for mapping various data source to the genome. Column one gives the source of the evidence and columns two and three give the total number of sequences in that set (as of May 2003) and the number of sequences that mapped with 95% identity and 50% coverage respectively. The SwissProt data was mapped using the GeneWise tool. The mapping rate can be considered to indicate the quality of the data and can be translated to a confidence metric level. The mapping rates of full-length cDNA sequences from the Mammalian Gene Collection (MGC) and curated NMs from NCBI's Reference Sequences are much better than those from GenBank mRNAs and ESTs. Approximately 99% of the sequences from MGC and RefSeq mapped onto the finished human genome, whereas only 86% of GenBank mRNA sequences and about 81% of EST sequences mapped on the genome. Approximately 20% (1 million) of the ESTs could not be mapped onto the genome. This is either likely due to the noisiness of the data or because, they were located in genomic repeat regions, or they had lower mapping qualities (less than 95% identity and 50% coverage). In this example, unmapped sequences were discarded.

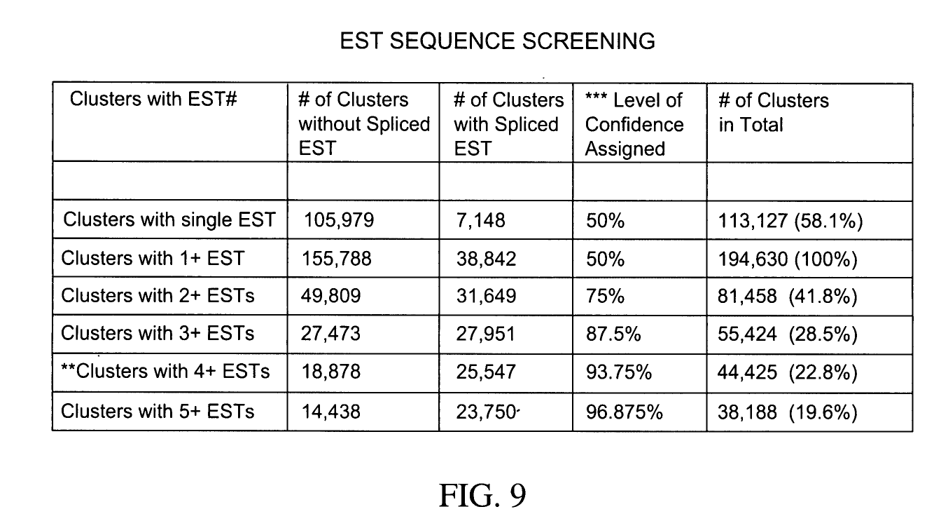

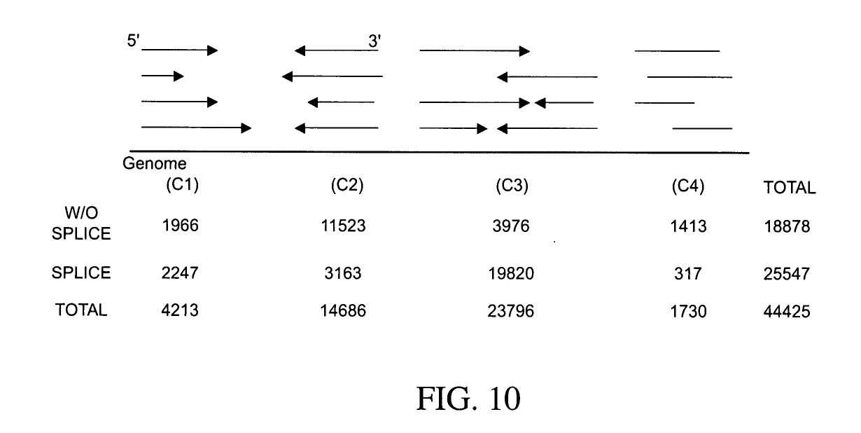

FIG. 9 illustrates how some embodiments collapse EST data into EST clusters and assign a confidence value to these clusters based on the number of EST sequences a cluster contains. This can be useful to overcome the inherent noisiness of EST data as correspondence between ESTs likely indicates that their sequence is correct. One method of calculating a confidence value for an EST cluster is to use the equation levelOfConfidence=(1-0.5\hat{ }n)*100% where n equals the number of ESTs in an EST cluster. Thus a cluster with a single EST has a confidence value of 50% while a cluster with 4 ESTs has a confidence of 93.75%. Some embodiments only retain clusters with four or more ESTs and refer to them as Highly-Confident Clusters. In this example, clusters with four ESTs are referred to as HC4-EST clusters. The genome was used as a reference to cluster related ESTs that were originally from the same genomic locations (loci) and the same strands. Collapsing selected EST sequences on the finished human genome resulted in forming about 194 K EST clusters, among which 113 K (58%) were singleton ESTs. 106 K (94%) of these singleton ESTs were not spliced, which may indicate genomic contamination. Results for this process are given in FIG. 9 which shows that 44,425 HC4-EST clusters were defined on the genome. Further analysis shows the distribution of the clone end types from these clusters (FIG. 10) where 33% of clusters only contained 3′-end EST (C2), and about 54% of clusters were mixtures of ESTs with either 5′ or 3′ ends (C3), whereas 9.5% of clusters only had 5′-end ESTs (C1), and less than 4% of HC4-EST clusters contained ESTs without clone end information specified (C4). Together with C2 and C3, 38,482 (87%) of HC4-EST clusters had ESTs with 3′ clone ends, which suggest that these transcribed gene loci could be confirmed. Such information is particularly useful for probe design in microarrays and gene expression assays.

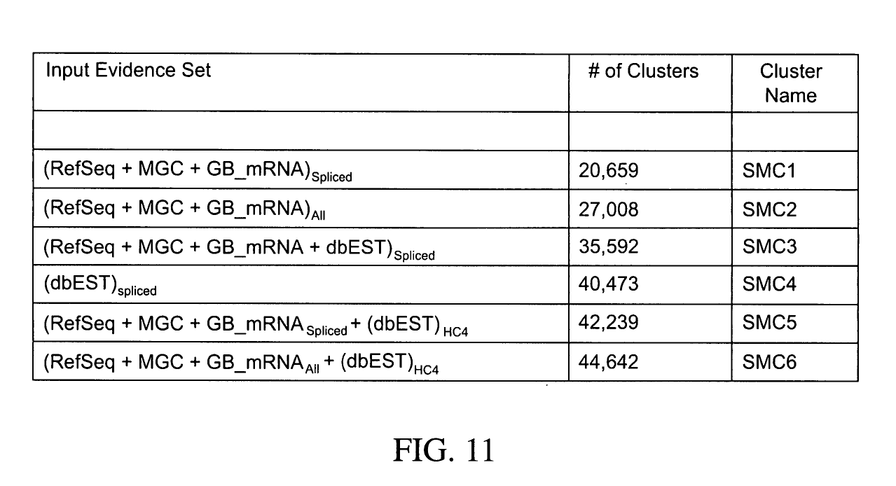

FIG. 11 gives the number of transcribed genes found using various data sets as evidence. With all qualified sequences from MGC, GenBank mRNA, HC4-ESTs, and RefSeq NMs used as evidence, 44,642 transcribed genes were identified. However, if HC4-EST and spliced evidence from MGC, GenBank mRNA, and RefSeq were included for clustering, 42,239 transcribed genes were defined. 35,592 transcribed genes could be identified if only spliced evidence from MGC, GenBank mRNAs, ESTs, and RefSeq were used. The results suggest that the majority of those transcribed genes contain at least one spliced expressed evidence sequence.

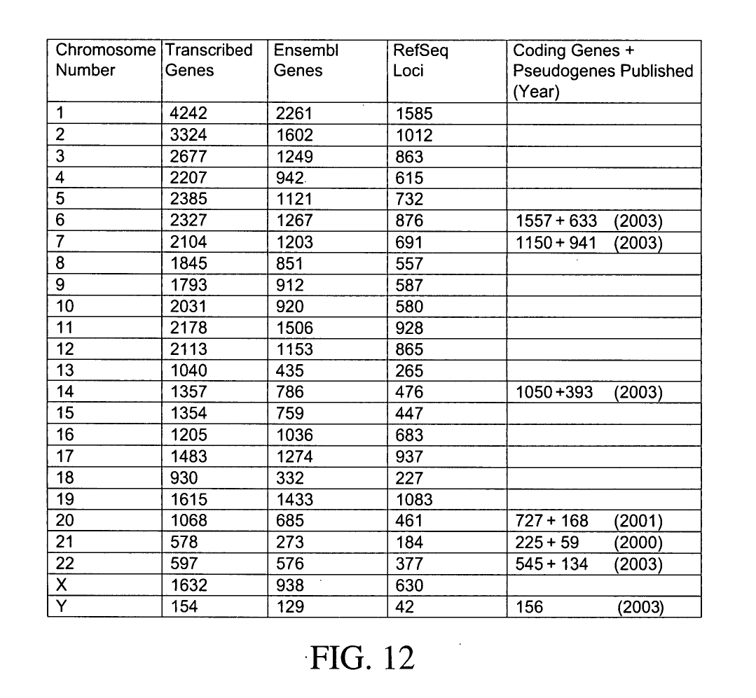

For comparison purposes, the numbers of transcribed genes for individual chromosomes, together with the numbers of Ensembl genes, RefSeq Loci, annotated by various groups since 2000 are listed in FIG. 12.

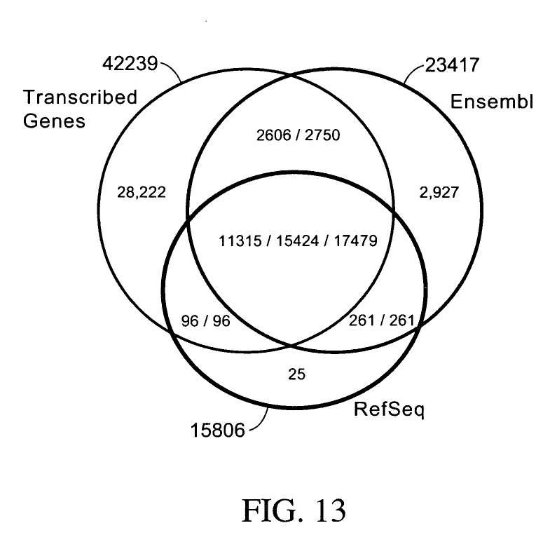

In FIG. 13, the number of transcribed gene loci (42,239) identified is much higher than the number found by Ensembl (23,417 genes). Further analysis revealed that 17,479 (75%) out of 23,417 genes from Ensembl corresponded with 15,424 (97.6%) out of 15,806 RefSeq gene loci and 11,315 (27%) out of 42,239 transcribed gene loci that were identified. The correspondence between these groups is contained in the overlapping region of the Venn diagram. In addition, 2,606 transcribed gene loci fell into gene locations occupied by 2,750 Ensembl genes without any RefSeq sequences in those regions. Therefore, 86.4% of Ensembl genes were included in the transcribed gene set, while only 3,188 (13.6%) of genes from Ensembl were not covered by the transcribed gene sets. It should be pointed out, however, that Ensembl combined ab initio genes with some protein evidence that were not included in this analysis. Moreover, the numbers of transcribed genes on chromosome 6, 7, 14, 20, 21, 22, and Y for which gene annotations have been completed (see last column in FIG. 12) were also higher than those published (as of December of 2003). This suggests, that a significant number of genes supported by experimental evidence were missed by previous genome annotation methods.

### EXAMPLE 2

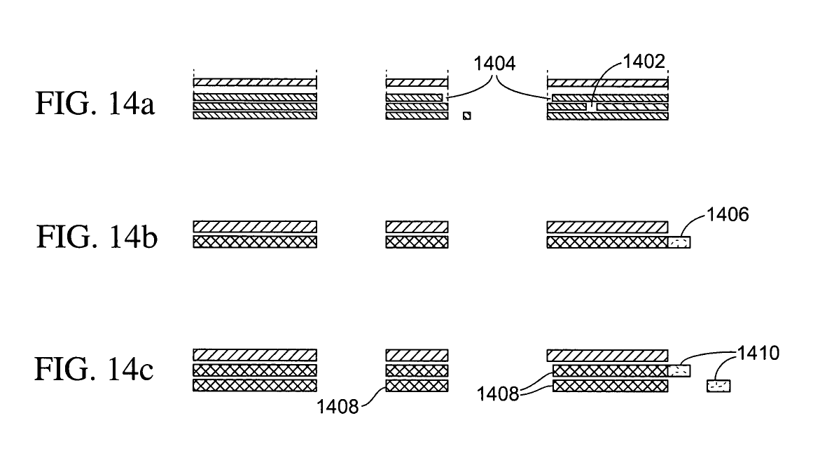

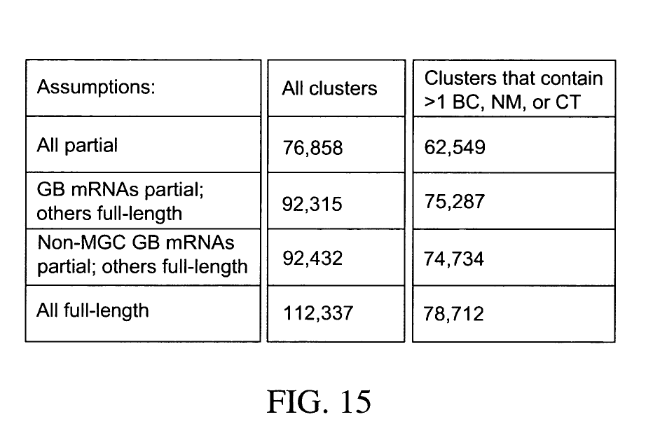

The rules for splice variant analysis were encoded in an automated computer pipeline. Prior to clustering the data, the rules were verified by passing the human Celera Transcripts (hCTs) through the pipeline. Because Celera Transcripts have undergone human curation, they are of high quality and the number of times Celera Transcripts cluster together should be low. The 62,472 hCTs that successfully mapped to the genome clustered into 59,977 transcript clusters. 2599 of these clusters contained more than one hCT, of which there were a total of 2977 total hCT pairs. Each of these pairs was grouped according to the structural differences they possessed. 264 pair had significant structural differences such as a missing exon or intron of less than twenty base pairs or where a splice site varied by more than twenty base pairs (1402 and 1404 on FIG. 14b respectively). 722 cases had perfect alignment at all splice sites and had extension of the 5′-most and/or 3′-most exon (1406 on FIG. 14b), which is allowed by the clustering rules. 1991 cases had small variations at the splice sites (less than 20 bp) (1408) and had extension of the 5′-most and/or 3′-most exon (1410) (FIG. 14c), this is also permitted by the rules. Although a cut-off of fifteen base pairs was used in the final analysis, values of ten and twenty were also used and yielded similar results. Because only 264 out of 62,472 hCTs were clustered in a way not allowed by the rules, the clustering error rate is approximately 0.4%.

Transcript clustering was attempted under 4 different sets of assumptions about sequence length. These are:
1. That no sequences could be trusted to be full-length. This zero-assumption of all sequences being partial (“All partial”) requires that a novel set of introns be present each time a new transcript cluster/structure is created.
2. That all sequences are full length (“All full-length”).
3. That GenBank mRNAs (including MGCs) are partial-length and RefSeqs and hCTs are full-length (“GB mRNAs partial; others full-length”).
4. That all non-MGC GenBank mRNAs are partial and that MGCs, RefSeqs, and hCTs are full-length (“Non-MGC GB mRNAs partial; others full-length”).

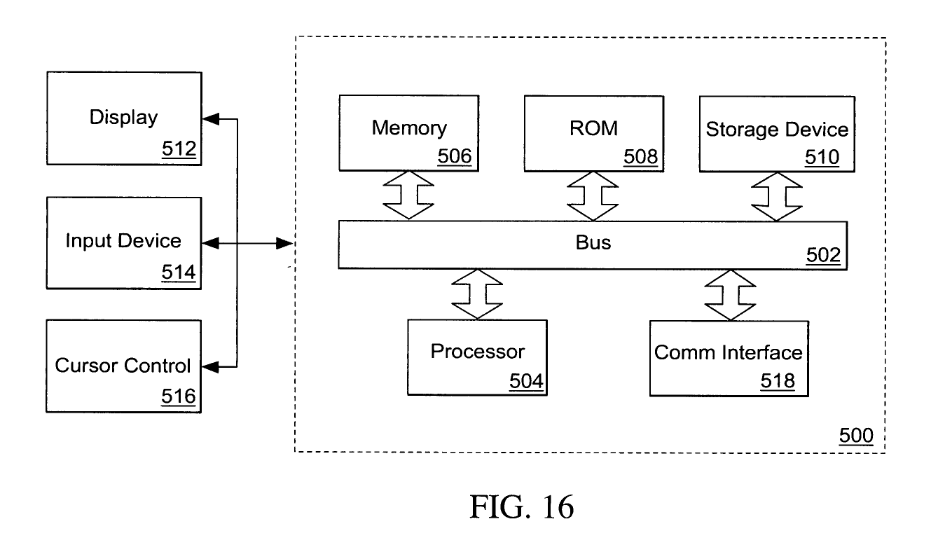

Results for these runs are contained in FIG. 17. The number of transcripts (clusters) identified varies from 76,858 to 112,337. Because of the lack of quality control on non-MGC GenBank mRNAs, the 112k number may not be very reliable. Most biologists strongly rely on the quality of RefSeq and Celera Transcripts. If these latter two are considered to be full-length, then 92,315 transcript clusters result. Assuming that MGCs are full-length adds very few additional clusters (92,432). 92 k transcripts is far more than is known by biologists.

Computer System Implementation

FIG. 17 is a block diagram that illustrates a computer system 500, according to certain embodiments, upon which embodiments of the present teachings may be implemented. Computer system 500 includes a bus 502 or other communication mechanism for communicating information, and a processor 504 coupled with bus 502 for processing information. Computer system 500 also includes a memory 506, which can be a random access memory (RAM) or other dynamic storage device, coupled to bus 502, and instructions to be executed by processor 504. Memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT) or liquid crystal display (LCD), for displaying information to a computer user. An input device 514, including alphanumeric and other keys, is coupled to bus 502 for communicating information and command selections to processor 504. Another type of user input device is cursor control 516, such as a mouse, a trackball or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Consistent with certain embodiments of the present teachings functions including evidence input, filtering, mapping, clustering and output can be performed and results displayed by computer system 500 in response to processor 504 executing one or more sequences of one or more instructions contained in memory 506. Such instructions may be read into memory 506 from another computer-readable medium, such as storage device 510. Execution of the sequences of instructions contained in memory 506 causes processor 504 to perform the process states described herein. Alternatively hard-wired circuitry may be used in place of or in combination with software instructions to implement the invention. Thus implementations of the present teachings are not limited to any specific combination of hardware circuitry and software.

The term “computer-readable medium” as used herein refers to any media that participates in providing instructions to processor 504 for execution. Such a medium may take many forms, including but not limited to, non-volatile media, volatile media, and transmission media. Non-volatile media includes, for example, optical or magnetic disks, such as storage device 510. Volatile media includes dynamic memory, such as memory 506. Transmission media includes coaxial cables, copper wire, and fiber optics, including the wires that comprise bus 502. Transmission media can also take the form of acoustic or light waves, such as those generated during radio-wave and infra-red data communications.

Common forms of computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red-signal. An infra-red detector coupled to bus 502 can receive the data carried in the infra-red signal and place the data on bus 502. Bus 502 carries the data to memory 506, from which processor 504 retrieves and executes the instructions. The instructions received by memory 506 may optionally be stored on storage device 510 either before or after execution by processor 504.

The foregoing description has been presented for purposes of illustration and description. It is not exhaustive and does not limit the invention to the precise form disclosed. Modifications and variations are possible in light of the above teachings or may be acquired from practice. Additionally, the described implementation includes software but the present teachings may be implemented as a combination of hardware and software or in hardware alone. The present teachings may be implemented with both object-oriented and non-object-oriented programming systems.

1. A method for splice variant identification comprising,
receiving genomic sequence information,
mapping said genomic information to a genome,
identifying overlapping overlapping mapped sequences,
identifying splice junction information in said overlapping mapped sequences,
forming clusters based on said splice junction information.
